(19) United States
(12) Patent Application Publication
(10) Pub. No.: US 2007/0281997 A1
(43) Pub. Date: Dec. 6, 2007

(54) TREATMENT OF HOT FLASHES USING MUSCARINIC RECEPTOR ANTAGONISTS

(76) Inventor: Joseph W. Aquilina, Easton, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/753,642

(22) Filed: May 25, 2007

Related U.S. Application Data
(60) Provisional application No. 60/803,644, filed on Jun. 1, 2006.

(51) Int. Cl. A61K 31/24

(52) U.S. Cl. 514/540

(57) ABSTRACT

The present invention relates to a method of treating hot flashes by administering a muscarinic receptor antagonist. The method is useful for treating men with prostate disorders being managed with androgen deprivation therapy.

### CROSS REFERENCE TO RELATED APPLICATIONS

This application claims the benefit of U.S. Provisional Application 60/803,644 filed on Jun. 1, 2006 which is incorporated by reference herein in its entirety.

### FIELD OF THE INVENTION

The present invention relates to the use of muscarinic receptor antagonists for the treatment of hot flashes (also referred to herein as hot flushes) in men. More particularly, the invention relates to the use of immediate and extended release formulations of these agents for the treatment of hot flashes in men with prostate disorders being managed with androgen deprivation therapy.

### BACKGROUND OF THE INVENTION

About 40-75% of men receiving androgen deprivation therapy experience hot flashes. Men who experience hot flashes describe physical and emotional distress, including feelings of anxiety, irritability and being out of control. The symptoms can range from mild to extremely severe and can interfere and interrupt normal daily activities, including sleep. This well-documented condition can significantly affect quality of life but has received little attention in men.

Androgen deprivation therapy has been the cornerstone of treatment for advanced prostate cancer since Huggins and Hodges first showed the dependence of prostate cancer on androgens in the early 1940's. They described hot flashes in nine of 21 castrated patients, starting 2-3 weeks after surgery. Huggins C., et al., “Studies on prostate cancer. I. The effects of castration, estrogen and androgen injections on serum phosphatases in metastatic carcinoma of the prostate”, Cancer Res, 1941: 1:293-5; Kouriefs C., et al., “Hot flushes and prostate cancer: pathogenesis and treatment”, BJU International, 2002, 89: 379-383.

The etiology of hot flashes in men receiving androgen deprivation therapy is also not fully understood. However, as the application of this treatment expands beyond prostate cancer, the treatment group continues to grow in both number and importance. Today, in addition to its established role in managing patients with metastatic disease, androgen deprivation therapy (including LH-RH analogues, antiandrogens, etc.) is often used to treat patients with increasing prostate specific antigen (PSA) levels after local prostate treatment with radiation or subsequent to surgical removal of the prostate. Even in the absence of additional evidence of metastatic disease, androgen deprivation therapy is also used as adjunctive therapy for men undergoing radiation therapy for high-risk localized disease (e.g. positive surgical margins, large bulky disease, high Gleason score tumors, etc.).

In men receiving androgen deprivation therapy, there are few data available regarding safe and effective therapies to reduce hot flashes. Previous reports have shown that hormonal agents such as megasterol are efficacious. However, there is some evidence suggesting that use of progestational agents, such as megesterol, may correlate with an increasing PSA level and/or increased risk of tumor progression. Burch, P., et al., “Prostate specific antigen decline after withdrawal of low-dose megesterol acetate”, [letter]. J Clin Oncol. 1999; 17:1087-1088. Antidepressants such as paroxetine and venlafaxine have shown efficacy in pilot studies; however, larger studies designed to more precisely define the optimal dosing regimen and safety/efficacy profile in men have not yet been reported. Quella S., et al., “Pilot evaluation of venlafaxine for the treatment of hot flashes in men undergoing androgen ablation therapy for prostate cancer”, J. Urology, 1999, July; 162(1):98-102; Loprinzi, C., et al., “Pilot evaluation of paroxetine for treating hot flashes in men”, Mayo Clinic Proc. 2004; 79(10):1247-1251.

It has been found that muscarinic receptor antagonists can effectively treat hot flashes in men with only limited side effects.

### SUMMARY OF THE INVENTION

The present invention provides a method for treating hot flashes in a subject in need of such treatment by administering a therapeutically effective amount of a muscarinic receptor antagonist. The invention is useful in treating men with prostate disorders being managed with androgen deprivation therapy. Accordingly, the present invention is useful in treating men with prostate cancer and men with increasing prostate specific antigen (PSA) levels after local treatment. The present invention is also useful in treating hot flashes in men where androgen deprivation therapy is used as adjunctive therapy for patients undergoing radiation therapy for high-risk localized disease (e.g. positive surgical margins, large bulky disease, high Gleason score tumors, etc.).

Muscarinic receptor antagonists useful in practicing the present invention include oxybutynin, tolterodine, trospium, darifenacin, solefenacin, hyoscyomine and combinations of these antagonists. Pharmaceutically effective salts or esters of these antagonists may be utilized, and where the antagonist exists as a racemate, individual enanteomers or a racemaic mixture of the enanteomers may be employed. The muscarinic receptor agonist may be provided either as an immediate release or extended release formulation and is administered via any route typically used for the administration of such agents, including oral, transdermal, topical, buccal, sublingual or microinjection administration.

Preferably, the muscarinic receptor antagonist is oxybutynin In the most preferred embodiment of the invention oxybutynin is administered via an extended release formulation, either orally or transdermally.

### DETAILED DESCRIPTION OF THE INVENTION

The efficacy of muscarinic receptor antagonists for the treatment of hot flashes in men with prostate disorders is determined via a double-blind, randomized, placebo-controlled safety and efficacy study in 12 to 20 subjects using DITROPAN XL as the study drug. The study includes men with a history of prostate cancer on a stable regimen of androgen deprivation therapy (including an LH-RH analogues and/or bilateral orchiectomy) who have bothersome hot flashes (defined as the occurrence of at least 14 episodes per week of sufficient severity to make the patient desire therapeutic intervention). Efficacy endpoints include the change in daily frequency of moderate to severe hot flashes from baseline and change severity of moderate to severe hot flashes from baseline. Safety assessments include pre- and post-study physical examination, laboratory analysis, adverse events, and vital signs including sitting pulse, blood pressure, and weight. Additional criteria for evaluation include scores from the Profile of Mood States (POMS), Pittsburgh Sleep Quality Index (PSQI), Menopause-Specific Quality of Life Questionnaire (MENQOL), Short Form-36 Health Survey (SF-36), Sleep Disruption Scale, and Subject Global Assessment.

Example I is a case study of a patient with a history of prostate cancer who was receiving androgen deprivation therapy. The case study demonstrates the efficacy of muscarinic receptor antagonists in treating hot flashes in such patients.

### EXAMPLE I

The patient is a 72 year old male with a history of prostate cancer (status post radical prostatectomy) who was receiving androgen deprivation therapy with an LH-RH analogue. The patient complained of approximately 20 hot flashes daily since starting androgen deprivation therapy, with an average day consisting of 1-2 severe, 4-5 moderate, and 14-15 mild episodes. He was started 10 mg Ditropan XL daily and experienced a marked reduction in both frequency and severity of hot flashes, where an average day consisted of 0 severe, 2-3 moderate, and 5-7 mild episodes. Because he experienced an occasional sensation of dry mouth, his dose of Ditropan XL was reduced to 5 mg of daily with a similar improvement in hot flash frequency and severity.

Various modifications of the invention in addition to those shown and described herein will be apparent to those skilled in the art from the foregoing description. Such modifications are also intended to fall within the scope of the appended claims. The cited patents or publications may provide further useful information and, accordingly, these cited materials are incorporated herein in their entirety by reference.

We claim:

1. A method for treating hot flashes in a subject in need of such treatment by administering a therapeutically effective amount of at least one muscarinic receptor antagonist, or a salt, ester, racemate or enanteomer of the antagonist.

2. The method of claim 1, wherein the subject is a male with a prostate disorder being managed with androgen deprivation therapy.

3. The method of claim 1, wherein the prostate disorder is selected from the group consisting of prostate cancer, where the treatment includes primary or adjunctive androgen deprivation therapy.

4. The method of claim 3, wherein the prostate cancer is organ confined prostate cancer.

5. The method of claim 4, wherein the prostate cancer is metastatic prostate cancer.

6. The method of claim 1 wherein the muscarinic receptor antagonist is administered in an extended release formulation.

7. The method of claim 1, wherein the muscarinic receptor antagonist is oxybutynin.
